(19) United States
(12) Patent Application Publication
(10) Pub. No.: US 2024/0285426 A1
(43) Pub. Date: Aug. 29, 2024

(54) DESIGN METHOD OF AN INTRAORAL DEVICE

(71) Applicant: BIOTECHNOLOGY INSTITUTE, I MAS D, S.L., Vitoria (Alava) (ES)

(72) Inventor: Eduardo ANITUA ALDECOA, Vitoria (Alava) (ES)

(21) Appl. No.: 18/566,761

(22) PCT Filed: May 17, 2022
(86) PCT No.: PCT/ES2022/070301

§ 371 Date: Dec. 4, 2023

(30) Foreign Application Priority Data
Jun. 7, 2021 (EP) 21382508.6

(51) Int. Cl. A61F 5/56; G06F 30/12

(52) U.S. Cl. CPC A61F 5/566; G06F 30/12; A61F 2005/563

(57) ABSTRACT

Design method of an intraoral splint-type device (1) to treat bruxism and/or sleep apnoea among others, using CAD/CAM software technology, where the method of the invention starts with an intraoral scan and is subsequently characterized by converting the arch (2, 3) defined as a one-piece mesh, into as many pieces as dental pieces (4) needed to be discerned, by means of thermal simulation. This segmentation makes it possible to isolate the geometry of each dental piece (4) in order to design the intraoral device (1) around each dental piece (4). In other words, it allows for the modification of the thickness (6c), height, etc. in a non-uniform way for each dental piece (4) according to the needs of the patient. This method allows the user very precise manipulation of the intraoral device (1) that is created, satisfying the mechanical and dental needs of each dental piece (4), and providing greater comfort to the patient.

### TECHNICAL FIELD

The invention refers to a method of designing an intraoral device through the use of a computer and a program or software for Computer-Aided Design, hereinafter CAD. Specifically, the method is for the design of intraoral devices, or oral splints, which are intended to be placed in the mouth of a user for the treatment of various jaw disorders such as bruxism, oral breathing or snoring and/or sleep apnoea, among others.

### STATE OF THE ART

Currently, various intraoral or mandibular devices for mandibular release and/or advancement are known to treat bruxism and sleep apnoea during the patient's sleep.

These mandibular devices are made to meet the specific needs of the patient and are known as discharge or mandibular advancement splints. On the one hand, mandibular discharge splints are devices made of a non-metallic, rigid material that is placed in the patient's mouth, generally in the upper arch, and they prevent the upper dental pieces from being pressed against the lower dental pieces. Moreover, a splint keeps the maxillary bones (upper and lower) in the correct position so that they do not exert more force than necessary during the time the splint is in place, making the muscles relax and preventing them from tensing.

On the other hand, these mandibular devices or mandibular advancement splints can also cause slight advancement of the lower jaw, which prevents airway closure while the patient sleeps. This device is also made to measure for the patient, and is placed inside the patient's mouth in such a way that it comprises an upper area intended to be placed in the upper dental arch and a lower area intended to be placed in the lower dental arch. The mandibular advancement mechanisms are connected between the upper zone and the lower zone, positioned and tensed in such a way as to keep the lower zone advanced with respect to the upper zone. This causes a slight advancement of the jaw with respect to the upper jaw, compared to a resting position in which both portions would be one on top of the other for the patient's normal bite. This opens a larger space at the back of the oral cavity and thus facilitates the passage of air to and from the pharynx.

The mandibular devices defined above are designed and manufactured using CAD/CAM technology; CAD being Computer Aided Design and CAM being Computer Aided Manufacturing. This technology is widely known in the dental sector, where normally any dental element such as a crown, implant or splint is designed using CAD and printed using CAM or 3D printing.

The design of mandibular devices or dental elements using this computer-implemented CAD/CAM technology begins with an intraoral scan of the patient. An intraoral scanner is a computer system where we enter the patient's data and the prosthetic prescription. Once the data has been entered, a fibre-optic device will be inserted into the patient's mouth and will take images of it until a complete 3D image is obtained. Together with the data, this 3D image forms a file that is sent to a design programme for personalized adaptation to each patient. The design of the dental elements is carried out using CAD software.

Currently, the design software known in the dental market is based on the design of mandibular devices through the generation and modification of meshes that define the mouth or dental arches of the patient. A mesh is a data set that defines the surface of the patient's arch in space as a single element. A dental arch is the set of incisor, canine and molar dental pieces that form the arch of the upper and lower jaws of a patient. The aforementioned design software works with a uniform thickness of the complete mesh, which will later be sent to 3D printing and be finished manually.

Once the dental product to be manufactured has been designed, a file is generated to send to the dental printer or 3D printer. In general, the 3D printing process is as follows: the printer, following the instructions from the design file, deposits and solidifies the material. Once the first layer has solidified, it continues with a second layer and repeats the same process, in such a way that the previously designed three-dimensional object is formed layer by layer. However, printed dental objects have been constructed of mesh with a uniform thickness, so that the object must be adjusted manually after printing, modifying and eliminating the excess parts of the material. This adjustment to adapt the device to the patient's mouth is very laborious, and is less detailed in the contact areas between the arches. In other words, the splint that is generated through the aforementioned processes is neither the most accurate nor the most comfortable for the patient.

This invention aims to provide an improved, more accurate method of designing a splint-type intraoral device that provides a more personalized design for greater patient comfort, in addition to minimizing manual adjustments after manufacturing. At the same time, this method of the invention provides the personalised treatment necessary to treat bruxism, sleep apnoea, snoring, tongue function or even create surgical guides.

### BRIEF DESCRIPTION OF THE INVENTION

The object of the present invention is an improved and more precise method of designing intraoral or mandibular devices, often called dental splints, to treat bruxism, oral respiration, known as snoring, lingual parafunction and sleep apnoea, among other disorders. Specifically, the implementation of the method of the invention will focus on the design of an intraoral device, or splint, without limiting the design method of the invention. The method of the invention is carried out through the use of CAD software installed on a computer, where firstly a file is created based on the intraoral or extraoral scan of the patient's previous models. The file contains the oral data of at least one arch or the previous models of the patient, and is transferred to the CAD software to visualize and work with at least one arch belonging to the patient, in the form of a mesh or single piece. Once the file is opened, the invention design method begins, characterized in that it first comprises a step where the arch mesh is segmented into various dental parts by means of thermal simulation. Thermal simulation consists of assigning heat points of one temperature to dental pieces and heat points of a different temperature to the dental pieces adjacent to the dental pieces with heat points of the first temperature. In this way, adjacent dental pieces have different heat points.

Once the heat points have been assigned to all the dental pieces by means of the CAD software, a thermal simulation is carried out by conduction, so that since heat is not transmitted between adjacent dental pieces as they are at different temperatures, all the dental pieces are defined separately.

Once each dental piece has been defined as individual pieces, the method of the invention continues to define local vectors and centroids to each dental piece, since the design of the device will be outlined one by one, so the local vectors will define the directions of a support curve for generating the final contour of the splint.

The method described has various advantages, such as identifying each and every dental piece identified dental piecein order to satisfy the mechanical and comfort needs of each dental piece for each patient. In addition, it gives more precise control over the design, such as being able to give non-uniform thicknesses throughout the splint. The possibility of designing the splint around each dental piece individually allows the contact surfaces between the opposing arches to be monitored and designed with greater precision and, by means of the design, to control the internal forces of the splint and prevent changes in the patient's bite in the future. In conclusion, the method of the invention makes it possible to design and manufacture personalized intraoral devices for each patient in a more precise way.

Additionally, thanks to the possibility of controlling the amount of material and thickness, it is also possible to adapt intraoral devices to products with less material, to facilitate 3D printing.

### BRIEF DESCRIPTION OF DRAWINGS

The details of the invention are shown in the following figures, which do not intend to limit the scope of the invention:

FIG. 1 shows an intraoral device (1), splint-type designed and manufactured according to the method of the invention.

FIG. 2 shows a patient's arches (2, 3) in the first step of the method of the invention.

FIG. 3 shows the second step of the method of the invention on an arch (2, 3) to be worked on.

FIG. 3a shows another view of the arch (2, 3) after the second step of the method of the invention.

FIG. 4 shows a view of the software in the third step of the invention.

FIG. 5 shows a view of the software at the start of the fourth step of the invention.

FIG. 6 shows a view of the software for the fourth step of the invention.

FIG. 7 shows a possible embodiment of the method of the invention.

FIG. 8 shows a view of the software for the fifth step of the invention.

FIG. 9 shows another possible embodiment of the method of the invention.

FIG. 10 shows a view after the sixth step of the invention.

FIG. 11 shows another possible embodiment of the method of the invention.

FIG. 12 shows another possible embodiment of the method of the invention.

### DETAILED DESCRIPTION OF THE INVENTION

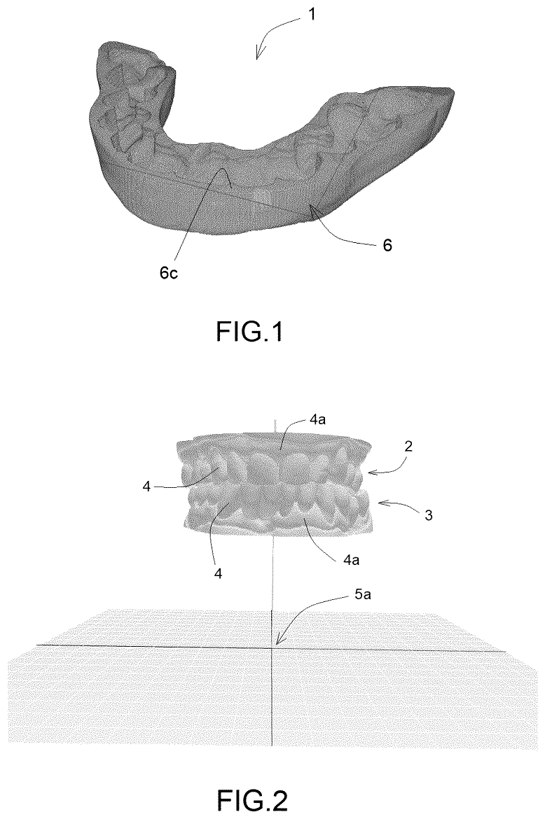

The invention refers to an improved and precise method for the design of intraoral devices (1), such as a dental splint or aligner, like the one seen in FIG. 1. Intraoral devices are aimed at dental treatment, where the most common are for the treatment of bruxism and/or sleep apnoea. As in other design methods, the method of the invention can also be applied to the design of other intraoral devices that are based on an intraoral scan of the patient or extraoral scan of the patient's previous models, the subsequent design of the device (1) using a computer and CAD software technology adapted to the patient's dental pieces and finally the 3D printing of the device (1) using CAM technology.

An intraoral scan is based on creating a 3D digital file of the patient's mouth and then working on it. Therefore, for the method of the invention, initially, an intraoral scan of the patient's mouth is carried out to generate at least one file with at least one arch (2, 3) of dental pieces to work with. Normally two files are generated separating the upper arch (2) and the lower arch (3) of the patient. Subsequently, the intraoral scan is transferred to a CAD program or design software using the arch file (2, 3) for which the splint-type intraoral device (1) design is to be configured, adapting it to the dental needs of the patient. As can be seen in FIG. 2, when the files are opened in the program, the patient's arch (2, 3) to be worked on is seen as a single piece or mesh with a uniform thickness that is established by the initial data as explained above.

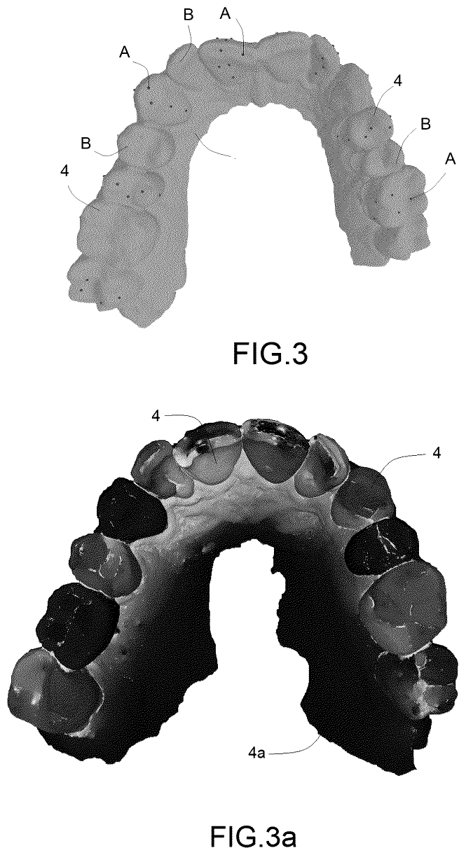

However, the method of the invention is characterized by segmenting the arch (2, 3) defined as a mesh or a single piece, into as many parts as dental pieces (4) that are to be worked on. The segmentation of the arch (2, 3) by means of the CAD software is carried out by means of a thermal simulation, by assigning heat points of temperatures A and B, in the different dental pieces (4) as seen in FIG. 3. Heat points of different temperatures are assigned, where certain heat points of one temperature (A) are assigned to some dental pieces (4) and other heat points of another temperature (B) to the dental pieces (4) adjacent to those that comprise the heat points of temperature (A). In this way, the adjacent dental pieces (4) are at different temperatures. In the CAD software, these heat points (A, B) are displayed in different colours, as seen in FIG. 3.

These heat points (A, B) are designed to segment the arch (2, 3) on which the work is carried out, defining the different dental pieces (4) of the arch (2, 3) by transmitting heat by conduction. Specifically, the thermal simulation is carried out using the software, and it is can be seen that the heat points (A, B) transmit heat through the mesh to a point that is defined by another temperature, as shown in FIG. 3a. In summary, since the adjacent dental pieces (4) comprise heat points (A, B) of different temperatures, the heat is transmitted to where the adjacent dental piece (4) begins, which is defined by another temperature. At that limit where heat is no longer transmitted is the border between the adjacent dental pieces (4), which corresponds to a minimum curvature area. The minimum curvature areas are found in the spaces between the dental pieces (4), and the dental pieces (4) and the gums (4a). In this way, it is possible to isolate the geometry of each dental piece (4) because heat is not transmitted between the different dental pieces (4).

This segmentation makes it possible to isolate the geometry of each dental piece (4) to be able to design the intraoral device (1) around each dental piece (4) individually in a non-uniform way according to the needs of each patient. This method allows the user a very precise manipulation of the intraoral device (1) to be created, satisfying the mechanical and dental needs of each dental piece (4) and providing greater comfort to the patient. In this way, an optimal design can be created to treat any jaw or dental disorder, such as bruxism, sleep apnoea, or to design aligners or surgical guides.

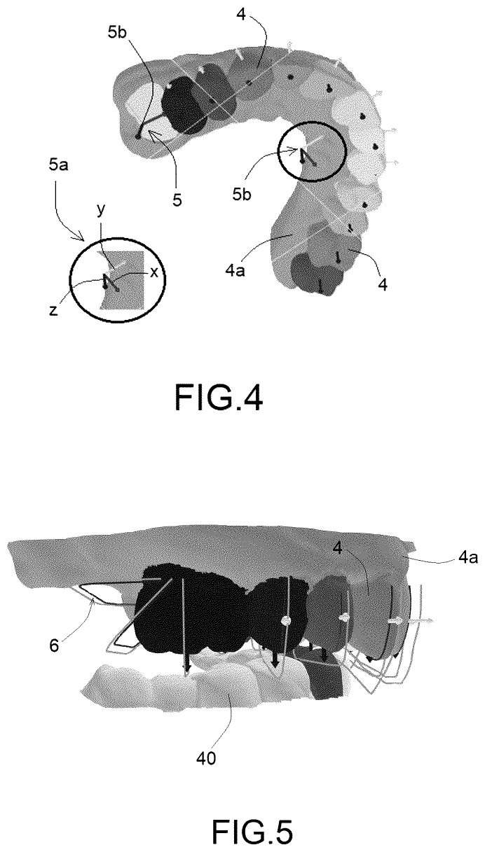

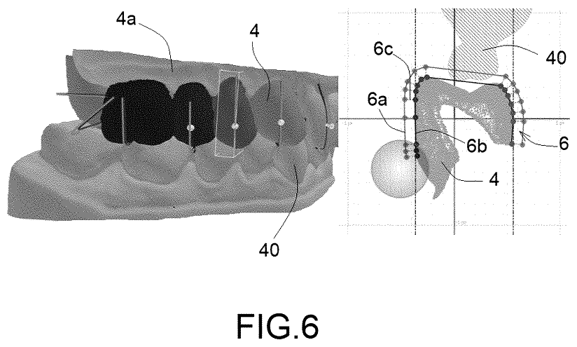

Next, once the dental pieces (4) have been separated, a 3D space is assigned to them, establishing vectors or local axes (5) and centroids (5b) which define the three directions (x, y, z) from the centroid (5b) of each dental piece (4) as seen in FIG. 4. A centroid (5b) is understood as the location of the geometric centre of gravity of a body. The local axes (5) of each dental piece (4) will be calculated according to the centroids (5b) of the adjacent dental pieces (4), the bisector between the adjacent centroids (5b) being the direction axis (Y) of each dental piece (4). A bisector is understood as the geometric place of the points of the plane originating at the vertex of an angle and equidistant from the sides of the angle, where the vertex is the centroid (5b) of the analyzed dental piece (4) and the sides of the angle are the lines that join the centroid (5b) of the analyzed dental piece (4) to the adjacent centroids (5b). The direction axis (X) is orthogonal to the direction axis (Y) and the direction axis (Z) is the same as that given in the initial data, i.e., the axis (Z) is parallel to the axis (Z) of the general axes (5a), as seen in FIG. 4. These axes (5) will be responsible for defining the directions of a contour (6) of the intraoral device (1) around each dental piece (4) as seen in FIGS. 5 and 6.

Next, as seen in FIGS. 5 and 6, in the region corresponding to each dental piece (4) a curve is defined that will serve as a support for the final contour (6) of the splint-type intraoral device (1) according to the method of the invention. The contour surface (6) of the intraoral device (1) is defined by an outer line (6a) defined by the vectors (5) and the surface curve of each dental piece (4) and can be designed and adapted independently. In the next FIG. 6, a sectional view of a dental piece (4) in the plane of directions (Y, Z) is shown. The outer line (6a) defines the outer contour (6) of the intraoral device (1) and an inner line (6b) defines the outer surface of the dental piece (4). The distance between the outer line (6a) and an inner line (6b) defines the thickness (6c) of the contour (6) of the intraoral device (1). In this way, each dental piece (4) will have a surface guiding curve, except for the last molars, which can rotate this direction plane (Y, Z) as many degrees as required to obtain the closure of the contour (6) of the intraoral device (1) as seen in FIGS. 5 and 6. This step allows each section that defines the thickness (6c) of the curve to be adapted and defined to determine the thickness (6c) of the intraoral device (1) dental piece by dental piece, adjusting the distances between the outer line (6a) and the inner line (6b) according to the mechanical or prosthetic needs of the patient. Furthermore, this step makes it possible to monitor the contact surface with the opposing dental piece (40) as shown in FIG. 6. This allows the generation of a splint type intraoral device (1), of non-uniform thickness (6c) in the different areas of the splint (1) according to dental diagnosis and treatment with infinite possibilities.

The method of the invention makes it possible to design and produce a splint type intraoral device (1), using independent geometries of the dental pieces (4), instead of using a mesh of the patient's arch (2, 3) as a single piece. In other words, the method generates a surface that is not forced to follow the geometry of the initial data. This highly personalized segmentation and adaptation of the contour (6) of the intraoral device (1) to each dental piece (4) is achieved thanks to the previous step of the method of the invention that has converted a mesh of the patient's arch (2, 3) into several independent dental pieces (4).

Optionally, it is even possible to eliminate the thickness (6c) completely in certain desired contact areas, according to design or dental criteria, so that the contact of the upper arch (2) with the opposing lower arch (3) is dental piece-splint and not splint-splint. This achieves an interrupted intraoral device (1) that provides greater comfort to the patient by reducing the total thickness (6c) of the intraoral device (1) placed in the patient's mouth. Additionally, if the patient is missing a dental piece (4) in one of their arches (2, 3), in the generation of the device (1) the missing piece is filled with material, so the thickness (6c) of the device (1) can be completely eliminated, since it includes additional material, to later carry out other necessary operations.

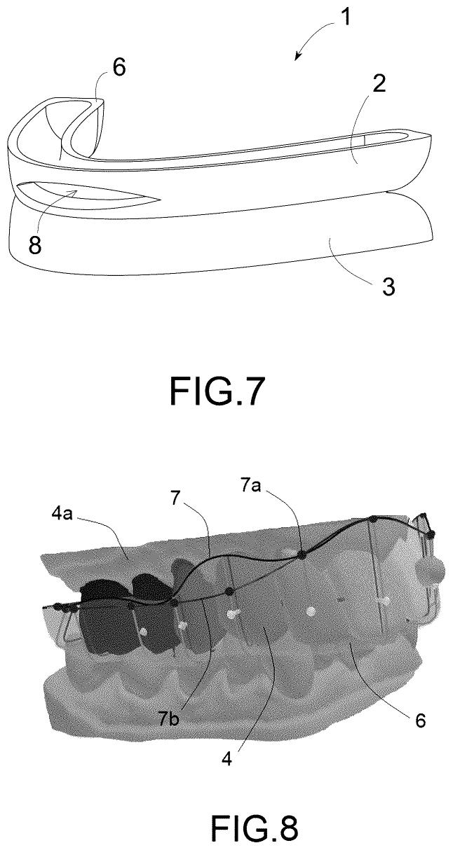

Optionally, FIG. 7 shows a possible embodiment of the method of the invention, which makes it possible to eliminate the thickness (6c) of the contour (6) in a frontal area, creating a frontal hole (8) where the incisor dental pieces meet. This front hole (8) makes it possible to reduce the load borne by the incisors, since the front hole (8) makes it possible to transfer this load towards the area of the root of the dental pieces (4). This achieves more comfort for the patient and reduces pain or possible future dental problems. This is an example of how the method of the invention makes it possible to control the action of the internal forces by means of the design of the intraoral device (1), preventing future changes in bite, or inducing them if this is the aim of the treatment.

Once the thicknesses (6c) of the intraoral device (1) have been defined for each dental piece (4) in the arch (2, 3), the next step of the CAD program is to generate a simulation of the final contour (6) of the intraoral device (1), which was defined in the previous step in which there is a limit curve (7) that represents the height of the intraoral device (1) as seen in FIG. 8. The next step is to modify the height of the final contour (6) of the intraoral device (1), i.e. defining the height of the intraoral device (1) with respect to each dental piece (4) by transferring some points (7a) on the limit curve (7), called a Spline in CAD design.

The height adjustment is carried out by transferring some points (7a) of the limit curve (7) or spline from each dental piece (4) to a desired height, defined by a final curve (7b) as shown in FIG. 8. It is possible to adjust the height of the points (7a) both in the vestibular area and in the lingual area (inside the mouth) to adjust the height of the intraoral device (1) with respect to the criteria or needs of each patient. The height of the splint is recommended to be above the equator of the dental piece to ensure minimal retention when placed in the patient's mouth. In this way, the height of the intraoral device (1) in each section of dental pieces or dental pieces (4) can be defined according to the criteria or dental or treatment needs of the patient.

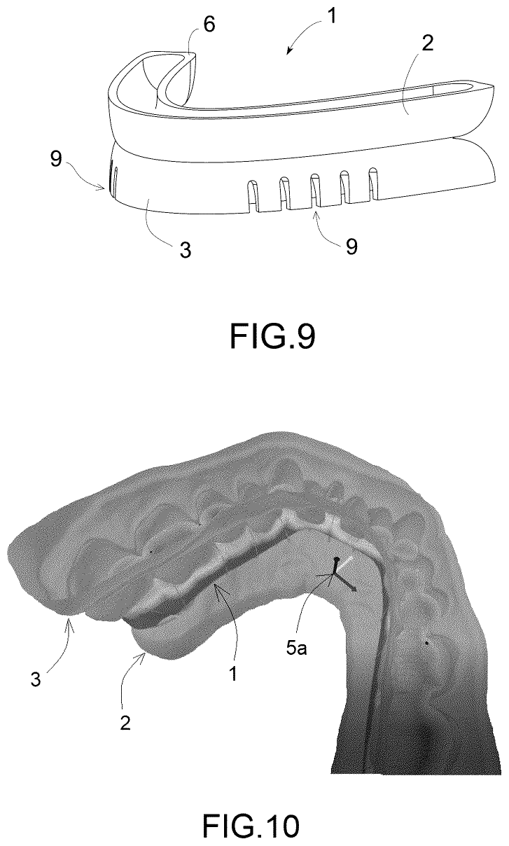

In addition, alternatively, another possible embodiment of the method of the invention consists of modifying the height of the intraoral device (1) in at least one interproximal area, defined as the space formed between the dental pieces, which is occupied by the gum (4a). This modification consists of moving the points (7a) of the limit curve (7) to a line below the equator of the dental piece, generating cuts or vertical grooves (9) to ensure the retention of the intraoral device (1) in the arch (2, 3) with a more comfortable insertion force, as shown in FIG. 9.

Once the thickness (6c) and height of the intraoral device (1) have been adjusted, the final contour (6) is generated in each dental piece (4) and the design made in the CAD program is accepted. Subsequently, all the fractions of the intraoral device (1), i.e. all the geometries of each dental piece (4), are joined together to convert the intraoral device (1) into a mesh again and work from now on with the intraoral device (1) as a whole. Once the mesh of the intraoral device (1) has been generated, the intersection of the arches (2, 3) with the opposing arch (2, 3) is calculated, to be able to define the amount of material desired in the contact areas between the arches (2, 3) according to the needs of the patient.

As can be seen in FIG. 10, the next step of the method, after converting the intraoral device (1) into a mesh, is to map the intraoral device (1) to visualize the contact areas between the arches (2, 3). Normally, in design software, the contacts between arches (2, 3) are defined with a gradient of colours defined by at least three shades according to the contact or intersection of the arches (2, 3) with the opposing arch. Generally, the colour gradient is red-orange-green according to the degree of intersection as shown in FIG. 10. In this way, it is possible to visualize where the amount of material in the contact areas that will bother the patient can be reduced or increased, simply by indicating the areas and choosing whether to add or remove material according to criteria established in the software tools. Through this step, it is possible to adapt the contact areas of the intraoral device (1) between the dental arches (2, 3), achieving the right occlusal adjustment without having to print and mould it manually.

Once the amount of material in the intraoral device (1) has been adapted to the prosthetic needs of the patient and the previous step has been validated, the penultimate step consists of performing Boolean operations. Boolean operations are defined as operations to create an object by combining two of them through a mathematical operation, where the two objects can be subtracted, joined or intersected to form the new object. In the design sector using CAD, this is a technique used in 3D, using planes, surfaces or solids to obtain volumes from the addition, subtraction or intersection of other volumes.

Optionally, a possible embodiment of the method of the invention by means of Boolean operations makes it possible to generate housings in the contact areas between arches (2, 3), similar to holes (12a) such as those seen in FIG. 12. Inside the aforementioned housings there are buttons made of resistant material such as titanium or wire. These buttons inserted into the intraoral device (1) allow the reinforcement of the areas where the housings are made, and provide greater hardness and resistance in the contact points or areas of strong bites between the arches (2, 3). In this way, instead of adding material or thickness (6c) to the contour (6), the intraoral device (1) obtains greater robustness with a lower thickness (6c) of the contour (6). In this way, the stiffness of the intraoral device (1) can be controlled and the result is a comfortable bite and a strong intraoral device (1). Alternatively, buttons made of elastic material can also be inserted into the housings for treatments that require greater flexibility in the bite. In short, these alternative operations make it possible to gain resistance and flexibility in the design.

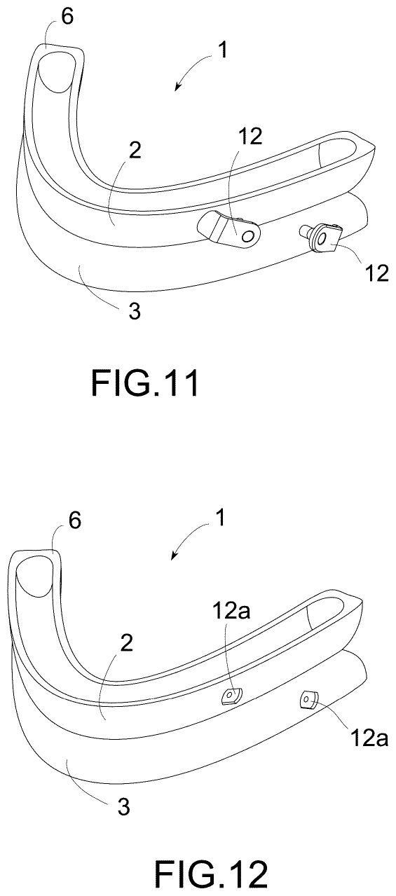

Additionally, for sleep apnoea treatments, it is necessary to connect mandibular advancement mechanisms, which are fixed to the sides of the upper (2) and lower (3) arches of the patient, positioned and tensed in such a way that they keep the lower arch (3) advanced with respect to the upper arch (2). Another possible embodiment of the method of the invention makes it possible to fix the mandibular advancement mechanisms or any other orthodontic system in the intraoral device (1) by generating projections (12) such as those shown in FIG. 11 or gaps (12a) intended to place adhesive as shown in FIG. 12, to hold the projections (12) as independent parts.

Another possible Boolean operation of the design method of the invention consists of generating guide holes in the splint-type device (1) in those areas of the splint where the patient is missing a dental piece (4). These holes intended to place a device that is metallic or of another material which serves for inserting and guiding the drilling tools used with a surgical guide, for example for the placement of an implant or other similar application.

Another possible Boolean operation of the method of the invention is based on generating a reticulated structure on the inner surface of the intraoral device (1). The internal reticulated structure is created from a geometric pattern on an YZ plane that is subsequently adjusted to the internal geometry of the intraoral device (1) through a transformation of curved coordinates. In this way, an intraoral device (1) is obtained with an irregular interior, with less rigidity, using less material and at the same time giving greater comfort to the patient.

Another possible Boolean operation of the method of the invention consists of selectively adding predefined protruding shapes to the splint (1), such as conical shapes, in the interior part of the upper or lower arch (2, 3) with certain functions, for example, educating the tongue so that it does not position itself or press in areas where it should not. In other words, the patient who presses or positions the tongue against the incisors, notices an uncomfortable surface, in such a way as to avoid that position. This makes it possible to prevent parafunctional habits, which are all jaw movements that have no functional purpose.

All these possibilities of the design method of the invention allow for the precise generation of an intraoral (1) or mandibular device for the treatment of each patient and with savings in material, since all corrections are made by CAD, facilitating 3D printing.

Finally, to complete the method and use of the CAD design program, the dental parts (4) are emptied to observe the intraoral device (1) as seen in FIG. 1 and the entry-exit path is defined for the intraoral device (1) placed in the patient's mouth, where the pathway may be irregular. In the case of the invention, it consists of generating the negative of the patient's arch (2, 3) in the intraoral device (1) to simulate the entry of the patient's real arch (2, 3) into the intraoral device (1), as would be done in the surgery by the dentist and the patient, so that the splint (1) can fit into the patient's mouth easily and comfortably.

It is also recommendable to perform a simulation of jaw movement to observe if the intraoral device (1) intersects or achieves the desired treatment. This simulation allows the design to be rectified if necessary before sending it to the 3D printer.

Once the design of the intraoral device (1) is finished using the computer CAD program, a file is generated with all the necessary data to be sent to the 3D printer or other CAM device, for the manufacture of the device (1).

1. A design method of an intraoral device, splint type, through the use of CAD software, where a file based on the intraoral scan or scan of the previous models of a patient is opened and the data of at least one arch of the patient in the form of a mesh or piece, where the design method is comprises the steps of:
segmenting the arch mesh into the different dental pieces by means of a thermal simulation by assigning heat points of a temperature to dental pieces and points of heat of another temperature to the dental pieces adjacent to those that comprise heat points of temperature, so that the adjacent dental pieces have different heat points,
defining at least some local vectors and centroids to each dental piece, which will define the directions of a support curve of a final contour of the intraoral device.

2. The design method of an intraoral device, according to claim 1, further comprising the additional steps of;
modifying at least one thickness of the contour of at least one dental piece individually,
generating a simulation of the final contour of the intraoral device, where a limit curve is generated to represent the height of the intraoral device in each dental piece,
modifying the height of the device by moving some points of the limit curve defined in each dental piece towards a final curve according to the prosthetic treatment needs of the patient.

3. The design method of an intraoral device, according to claim 1, wherein the segmentation of the mesh is carried out by transmitting heat by conduction to areas of minimum radii of curvature between the different dental pieces.

4. The design method of an intraoral device, according to claim 2, wherein the modification of the thickness comprises the additional step of adjusting the distance between an outer line and an inner line that define the thickness of each dental piece.

5. The design method of an intraoral device, according to claim 2, wherein the modification of the thickness comprises the additional step of completely eliminating the thickness in at least one contact area, so that the contact of the intraoral device of the upper arch with that of the opposing lower arch is dental piece-splint and not splint-splint.

6. The design method of an intraoral device, according to claim 2, wherein the modification of the thickness allows a front hole to be made in the front area of the intraoral device where the incisor dental pieces are located.

7. The design method of an intraoral device, according to claim 2, wherein the modification of the limit curve that defines the height of the intraoral device comprises the additional step of transferring the points from the limit curve to a final curve below the equator of the dental piece generating vertical cuts or grooves.

8. The design method of an intraoral device, according to claim 1, further comprising the additional steps of;
joining the individual geometries of each dental piece of the splint-type intraoral device to convert the final contour into a mesh or single piece,
removing or adding material from the final contour mesh of the splint-type intraoral device and,
generating the file with a splint-type device designed to send to the 3D printer or another CAM device, for manufacturing.

9. The design method of an intraoral device, according to claim 8, wherein additional Boolean operations are performed during the step of adding or removing mesh material.

10. The design method of an intraoral device, according to claim 9, where the Boolean operation consists of creating housings where buttons of resistant material such as titanium or wire are fixed to provide greater hardness and resistance in the contact areas or areas of strong bites between the patient's dental arches with a lesser thickness of the device.

11. The design method of an intraoral device, according to claim 9, where the Boolean operation consists of creating housings where buttons of elastic material are attached for treatments that require greater flexibility in the bite of the patient's mouth and a lesser thickness of the intraoral device.

12. The design method of an intraoral device, according to claim 9, where the Boolean operation consists of generating projections in the splint to fix mandibular advancement mechanisms or other orthodontic systems.

13. The design method of an intraoral device, according to claim 9, where the Boolean operation consists of generating holes in the splint intended to place adhesive to hold the aforementioned projections as independent pieces, where the mandibular advancement mechanisms are fixed.

14. The design method of an intraoral device, according to claim 9, where the Boolean operation consists of generating guide holes in the splint-type device intended to place a metal device or other material that serves to guide the milling tools used with a surgical guide or other similar application.

15. The design method of an intraoral device, according to claim 9, where the Boolean operation consists of generating a reticulated structure on the inner surface of the splint-type device that is created from a geometric pattern on a YZ plane, which by a curved coordinate transformation is adjusted to the internal geometry of the splint-type device.
